(19) United States
(12) Patent Application Publication
(10) Pub. No.: US 2003/0042126 A1
(43) Pub. Date: Mar. 6, 2003

(54) Process and reactor design for the photocatalytic conversion of natural gas to methanol

(76) Inventors: Duyen T. Nguyen, Castro Valley, CA (US); Krishniah Parimi, Concord, CA (US)

Correspondence Address: James W. Ambrosius, Chevron Corporation, P.O. Box 6006, San Ramon, CA, 94583-0806, US

(21) Appl. No.: 09/944,804

(22) Filed: Aug. 30, 2001

(51) Int. Cl. B01J 19/08; C07C 29/00

(52) U.S. Cl. 204/157.9; 422/186

(57) ABSTRACT

A photocatalytic method and apparatus employing a Holl-type mill for the direct production of methanol from methane and water comprising forming a water/methane emulsion and contacting the emulsion with a photocatalyst under conditions to react the methane and water to form methanol.

### FIELD OF THE INVENTION

This invention relates to a process and a reactor design for the photocatalytic conversion of natural gas and water directly to methanol.

### BACKGROUND OF THE INVENTION

Methanol or methyl alcohol is useful as a fuel, as a solvent, in antifreeze, and as the starting material for the manufacture of other products. Although methanol may be produced by wood distillation, most methanol used today is manufactured by means of a synthetic route. In one synthetic route, natural gas, which is primarily methane, is converted to synthesis gas, also called syngas, which is a mixture of carbon monoxide and hydrogen. The carbon monoxide and the hydrogen are reacted over a catalyst, usually containing zinc oxide, at relatively high temperatures and pressure to produce methanol. This is a two-step process involving relatively severe reaction conditions, so a one-step synthetic route involving mild reaction conditions would be desirable. Although methanol is known to be a partial oxidation product of methane, no practical commercial route is available involving the direct conversion of methane to methanol. Most direct conversion processes which have been proposed in the literature produce unsatisfactory yields of methanol, making them commercially impractical.

One particularly interesting method for the direct conversion of methane to methanol uses a photocatalytic reaction. Kotaro Ogura and Makato Kataoka describe in the Journal of Molecular Catalysis, 43 (1988) pp. 371-379, a method for the direct conversion of a mixture of methane and water under mild conditions. In this process, Ogura and Kataoka used UV radiation to dissociate the water molecules into a hydroxyl radical and hydrogen. The free hydroxyl radical abstracts a hydrogen from the methane molecule forming a methyl radical. The methyl radical reacts with water to produce methanol and other oxygenates. The reaction conditions were mild, involving temperatures within the range of 50° C. to 90° C. and at atmospheric pressure. Unfortunately, the yield of methanol was very low.

It is known that UV radiation of early transition metal oxides, such as molybdenum oxide, titanium oxide, and vanadium oxide, in the presence of oxygen form O radical ion sites that actively abstract hydrogen from methane. See Kenji Wada et al., Applied Catalysts A: General, 99 (1993) pp. 21-36. Ward et al. in J. Phys. Chem., 1987, 91, pp. 6515-6521, reports that molybdenum trioxide doped with copper can be activated with visible light.

U.S. Pat. No. 5,720,858 covers a photocatalytic process for the direct conversion of methane to methanol with visible light using a semi-conductor catalyst and an electron transfer agent.

A significant limitation on all of the methods for direct conversion of methane described above involve the problem of contacting the reactants, i.e., methane and water. Most processes involve bubbling the methane through the water, generally with some kind of mechanical stirring. Since methane is not highly soluble in water and most of the conversion reactions must take place at the interface between the methane bubbles and water, the yields of methanol tend to be poor. Therefore, the relatively small reaction interface between the various reactants and the catalyst appears to be a critical limitation on the efficiency of the reaction.

In an attempt to improve the reaction interface between the methane and water, U.S. Pat. No. 6,129,818 describes a photocatalytic method and apparatus for converting methane and water to methanol using a rotating semi-permeable sintered stainless steel tube where the methane is introduced under positive pressure inside of the tube with the water being present on the outside. The methane penetrates the tube and forms small bubbles on the outside surface of the tube where they are sheared off and react with the water in the presence of a photocatalyst and UV light to form methanol.

The present invention is directed to a novel reactor design which is used to form an emulsion between the water and methane, greatly improving the interface between the methane and water. The methane and water emulsion is contacted under mild conditions with a photocatalyst in the presence of light to produce methanol and hydrogen.

### BRIEF DESCRIPTION OF THE INVENTION

In its broadest aspect, the present invention is directed to a photocatalytic method for the production of methanol from methane and water which comprises (a) forming an emulsion comprising a mixture of methane and water; (b) exposing the emulsion of methane and water in a photocatalytic reaction zone to light in the presence of an effective catalytic amount of a photocatalyst under conditions suitable to support the conversion of methane and water to methanol, whereby the methane and water react to form methanol; and (c) recovering methanol from the photocatalytic reaction zone. For the most efficient reaction conversion, it is advantageous to maintain the emulsion in a sufficiently narrow layer to allow the penetration of the light through its entire depth. As will be explained in greater detail below, the methane/water emulsion is most readily achieved by use of a Holl-type mill which employs the opposing movement of at least one of two mill surfaces relative to one another to form a high shear zone where sub-Kolmogoroff eddies are formed in the fluid mixture introduced into the space between the two mill surfaces. This embodiment also holds the emulsion in a relatively narrow layer facilitating good light penetration. Accordingly, a preferred embodiment of the method of the present invention may be described as a photocatalytic method for the production of methanol from methane and water which comprises (a) forming an emulsion comprising a mixture of methane and water using a Holl-type mill which is characterized by a high shear treatment zone that is created by the opposing movement of at least one of two mill surfaces relative to one another wherein sub-Kolmogoroff eddies are formed in the space between said mill surfaces; (b) exposing the emulsion of methane and water in a photocatalytic reaction zone to light in the presence of an effective catalytic amount of a photocatalyst under conditions suitable to support the conversion of methane and water to methanol, whereby the methane and water react to form methanol; and (c) recovering methanol from the photocatalytic reaction zone.

The present invention is also directed to a novel reactor design which incorporates a Holl-type mill. In its broadest aspect, this aspect of the invention may be described as a reactor suitable for carrying out a photocatalytic reaction between reactants in order to form a product, wherein at least one liquid reactant and at least one gaseous reactant which is insoluble in said liquid are present, said reactor comprising (a) a Holl-type mill which is characterized by a high shear treatment zone that is created by the opposing movement of two mill surfaces relative to one another wherein sub-Kolmogoroff eddies are formed in the space between said mill surfaces, whereby an emulsion comprising the liquid reactant and the insoluble gaseous reactant will be formed, and wherein at least one of the said mill surfaces will allow the passage of light into the space between said mill surfaces; (b) a light source positioned relative to at least one of said mill surfaces which admits light into the high shear treatment zone; (c) an effective catalytic amount of a photocatalyst located in the high shear treatment zone; (d) means for introducing a liquid reactant and a gaseous reactant into the high shear treatment zone; and (e) means for withdrawing product from the high shear treatment zone.

More specifically, the present invention is directed to a reactor suitable for the photocatalytic production of methanol from methane and water which comprises (a) a Holl-type mill which is characterized by a high shear treatment zone that is created by the opposing movement of at least one of two mill surfaces relative to one another wherein sub-Kolmogoroff eddies are formed in the space between said mill surfaces, whereby an emulsion comprising methane and water will be formed, and wherein at least one of the said mill surfaces will allow the passage of light into the space between said mill surfaces; (b) a light source positioned relative to at least one of said mill surfaces which admits light into the high shear treatment zone; (c) an effective catalytic amount of a photocatalyst located in the high shear treatment zone; (d) means for introducing methane and water into the high shear treatment zone; and (e) means for withdrawing methanol from the high shear treatment zone. As explained in greater detail below, the preferred configuration of the Holl-type mill employs two cylinders, one of which is located inside of the other. In this preferred reactor design, the two mill surfaces of the Holl-type mill comprise the inner surface of a hollow outer cylinder and the outer surface of an inner cylinder, wherein the two cylinders are longitudinally positioned relative to one another such that the annular space formed between said inner surface of the hollow outer cylinder and said outer surface of the inner cylinder form the high shear treatment zone and the high shear is created by the rotation of at least one of the cylinders about its longitudinal axis relative to the other cylinder.

### BRIEF DESCRIPTION OF THE DRAWINGS

FIG. 1 is a partially schematic perspective view from one side of a reactor utilizing the Holl-type mill design which also illustrates the cylinder within a cylinder configuration.

FIG. 2 is a partially schematic perspective view from one side of an alternate embodiment of the cylinder within a cylinder configuration.

### DETAILED DESCRIPTION OF THE INVENTION

In the present invention, the methane and water form an emulsion which is contacted with a photocatalyst in the presence of light, preferably light in the visible spectrum, to initiate the conversion of the reactants to methanol and hydrogen. The general reaction may be characterized as follows:

CH₄+H₂O→CH₃OH+H₂

While not wishing the present invention to be bound by any particular chemical mechanism, some theory of what is happening may be useful in understanding the invention. It is theorized that the photocatalyst, when exposed to light, initiates the dissociation of the water molecule into a free hydroxyl radical and hydrogen. The hydroxyl radical is believed to abstract a hydrogen from the methane, forming a methyl radical. The methyl radical then reacts with water to form methanol and free hydrogen.

The critical step in carrying out the process of the present invention involves the formation of the methane/water emulsion. As used in this disclosure, the term “emulsion” refers to a fluid consisting of a microscopically heterogeneous mixture of methane and water, in which the methane forms minute micelles within a continuous water phase. The actual chemical reaction that produces methanol takes place at the water-methane interface of the methane micelles. Since the methane micelles in the continuous water phase of the emulsion significantly increase the interface surface for the reaction to occur as compared to those processes described in the prior art, the conversion of methane and water to methanol will proceed much more efficiently than has hitherto been possible.

The preferred method for forming the gas/water emulsion uses what is referred to in this disclosure as a Holl-type mill. Holl-type mills are described in detail in U.S. Pat. No. 5,538,191, the entire contents of which are herein incorporated by reference. Several different Holl-type mill configurations are described in the patent. One configuration uses two circular plates which are mounted one above the other to form a high shear zone in the gap formed between the two plates. However, the preferred configuration of the reactor for the purpose of the present invention utilizes what is referred to herein as the cylinder within a cylinder configuration. In the preferred embodiment of the present invention, the Holl-type mill also serves as the reactor. Holl-type mills and Holl-type mill reactors are commercially available from Holl Technologies Company located in Ventura, Calif.

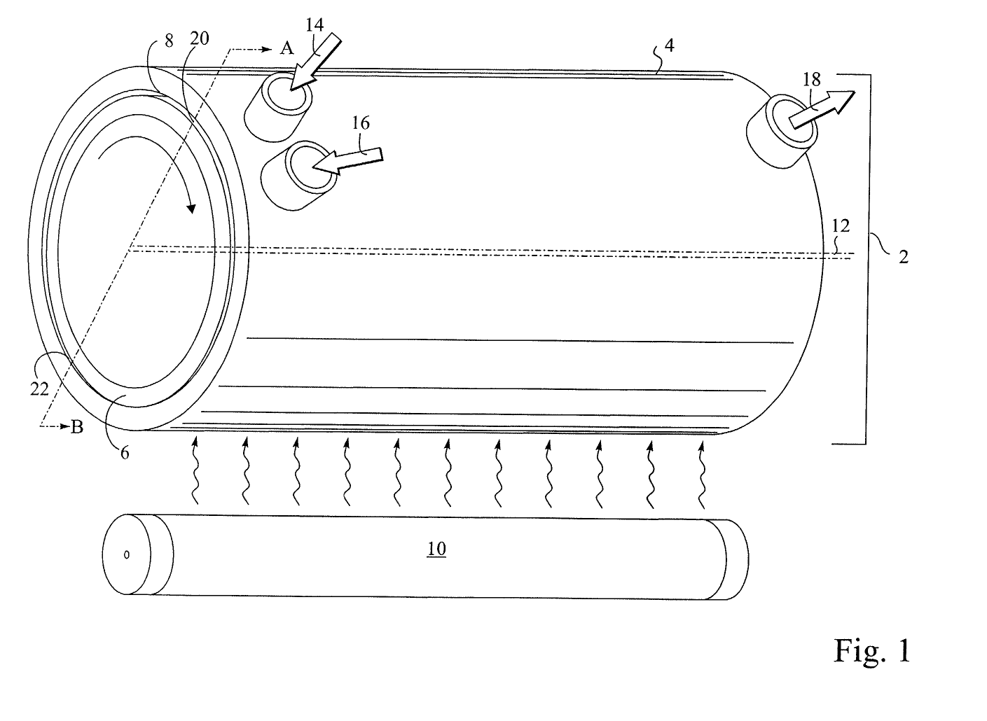

The operation of the cylinder within a cylinder Holl-type mill reactor may be more clearly understood by reference to FIG. 1. In this embodiment, the reactor 2 comprises a stationary outer cylinder 4 and a rotating inner cylinder 6. The inner cylinder is positioned somewhat asymmetrically within the outer cylinder so that the annular space 8 formed between the inner surface of the outer cylinder and the outer surface of the inner cylinder varies in width about its circumference. This will be more clearly understood by referring to line AB in the figure. It will be noted that the gap 20 in the annular space 8 at point A is significantly wider than the gap 22 in the annular space at point B of line AB. In this embodiment, the outer cylinder is made of a clear material, such as glass or acrylic, which admits light from a light source 10 into the annular space 8. The inner cylinder 6 rotates on its axis 12 within the outer stationary cylinder 4 and the relative motion of the inner and outer cylinders creates the high shear forces within the fluids present in the annular space. The outer wall of the inner cylinder is coated with an immobilized photocatalyst. The outer cylinder 4 has a methane inlet 14 and a water inlet 16 located on one end of the reactor. Although not shown as such in the schematic, preferably, the methane and water will be introduced tangentially into the most constricted point of the annular space to increase the shear. A discharge inlet 18 for the withdrawal of product and unreacted methane and water is located at the opposite end. In operation, methane enters the annular space 8 via inlet 14 and water enters via inlet 16. In the annular space, the water and methane mix to form an emulsion as a result of the eddies formed by the rotation of the inner cylinder 6 within the outer cylinder 4. The light source 10 produces visible light which penetrates the outer cylinder and activates the photocatalyst coating the outer wall of the inner cylinder. Since the emulsion formed by the water and methane will be opaque, the annular space must be sufficiently narrow that the photons from the light source can penetrate to the photocatalyst on the outer wall of the inner cylinder. Since the design of the Holl-type mill requires that the annular space be rather narrow in order to create the eddies responsible for the high shear, light penetration is not normally a problem with this reactor. The photocatalyst, when activated, catalyzes the conversion of methane with water to form methanol. The emulsion and methanol move the length of the annular space and are withdrawn via outlet 18. From outlet 18, the reaction mixture containing methane, water and methanol is sent to a separator (not shown), where the methanol is recovered separately from the methane, water and other oxygenated hydrocarbon by-products formed in the reactor. The water and methane may be recycled back to the reactor for further treatment if so desired.

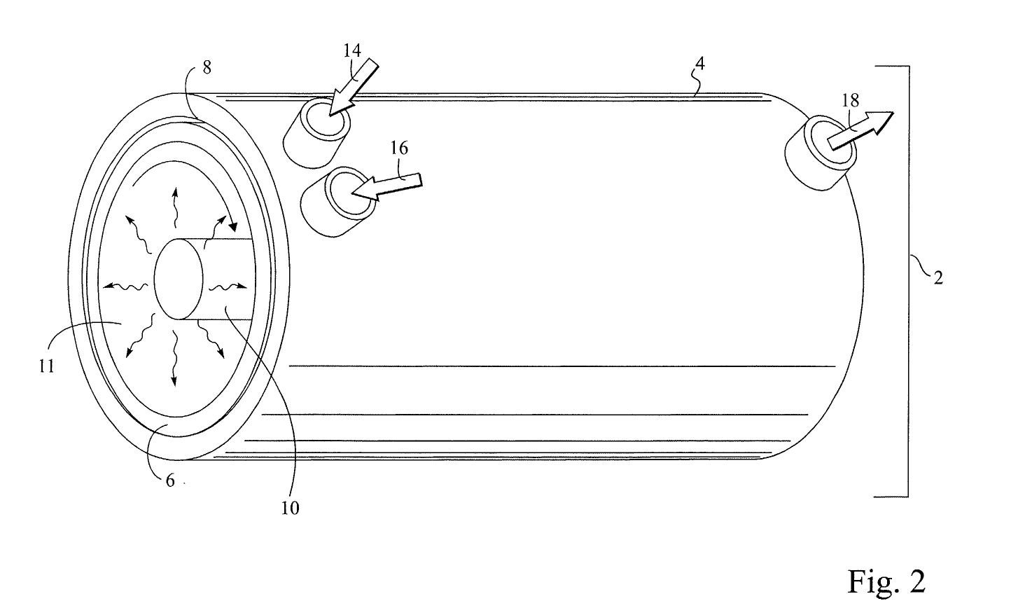

FIG. 2 represents an alternative embodiment of the cylinder within a cylinder Holl-type mill reactor. In this configuration of the invention, the light source 10 is located within the chamber 11 enclosed by the inner cylinder 6. This embodiment is advantageous in that almost all of the light generated by the light source 10 will be directed through the wall of the inner cylinder 6 and into the annular space 8 where it is available to activate the immobilized photocatalyst which is coated on the inner surface of the outer cylinder 4. Of course, in this embodiment, the inner cylinder must be fabricated from a clear material to allow the light to penetrate into the annular space 8.

The manner in which the methane/water emulsion is believed to be formed in the Holl-type mill is explained in detail in U.S. Pat. No. 5,538,191. In regard to the present invention, the precise mechanism of how the emulsion is produced is important only for the purpose of describing the characteristics that are unique to the Holl-type mill. The high shear which occurs in the annular space between the two cylinders is caused by the suppression of Kolmogoroff eddies in the narrowest portion of the annular space, i.e., that part of the annular space shown as gap 22 in the drawing. Dr. A. N. Kolmogoroff has shown that mixing of a liquid with an immiscible gas or solid depends upon the production of eddies within the fluid. Conventional mixers when water is the dispersion vehicle and at a temperature of 20° C. are unable to produce eddies of a diameter smaller than about 10 to 20 micrometers. In the specific embodiment of the invention when the emulsion is between water and methane, those methane bubbles which are entrained in the water and are of smaller size than the minimum size eddy (Kolmogoroff eddy) become part of these Kolmogoroff eddies and are, therefore, shielded against the effect of turbulence in the water. Consequently, additional mixing is ineffective below this limit. The Holl-type mill is designed to suppress the Kolmogoroff eddies in the narrowest portion of the annular space creating what is referred to as sub-Kolmogoroff eddies which are areas of turbulence smaller than the minimum size eddy normally achieved when water is the dispersion vehicle. These small sub-Kolmogoroff eddies appear to be responsible for the formation of the gas/water emulsions capable of being formed in the Holl-type mill. It should also be understood that, for the purposes of this disclosure, the high shear zone refers to the entire annular space between the two cylinders, not just to the narrowest portion where the sub-Kolmogoroff eddies are created. An additional advantage of the Holl-type mill reactor is that the turbulence results in efficient contacting between the catalyst and the reactants when the photocatalyst is present in the high shear zone.

In the two embodiments of the reactor shown in the drawings, at least one of the cylinders is clear in order to admit light into the annular space. Since the reactants are not abrasive and the reactor operates under relatively mild conditions with the pressure not exceeding about 20 atmospheres, and generally operating at about atmospheric pressure, and with the temperature not exceeding the boiling point of water, the selection of materials is not a significant problem. Glass or acrylic would be suitable for use in the fabrication of the clear cylinder. In the embodiment shown in FIG. 1, the photocatalyst is immobilized on the outer surface of the inner cylinder. Again, the materials of construction are not deemed critical other than that the outer surface of the cylinder must be suitable for immobilizing the catalyst. Generally, stainless steel would be entirely suitable for the fabrication of the cylinder on which the catalyst is immobilized. In the embodiment of the reactor shown in FIG. 2, where the light source is placed within the inner cylinder, the inner cylinder would need to be clear and the outer cylinder would immobilize the catalyst on its inner surface.

The light source may be any light source of the proper wavelength to activate the photocatalyst. Although the use of UV light has been described in the literature, it would be preferred that the light be within the visible range. This simplifies the reactor design and eliminates possible worker exposure problems associated with UV light. Natural sunlight, incandescent lamps, fluorescent lights, and the like are suitable light sources for use in the present invention. The intensity must be sufficient to penetrate the cylinder and the emulsion to activate the catalyst. Normally, this is not a problem when a Holl-type mill reactor is used, since the high shear zone is relatively narrow by design. Other than as noted, the exact placement of the catalyst within the reactor, the materials of construction, and the placement of the light source relative to the reactor are not critical to the operation of the invention and will be generally dictated by engineering design principles that are understood and well within the ability of a competent engineer.

Photocatalysts suitable for use in carrying out the present invention have been described in the literature. The photocatalyst must be capable of dissociating water into the hydroxyl radical and hydrogen under the conditions present in the reaction zone. Suitable photocatalysts include transitions metals such as tungsten, bismuth, ruthenium, iron, titanium, and cadmium. Particularly useful are molybdenum, vanadium, titanium, and tungsten, with tungsten and titanium being especially preferred. Usually, the metals will be present as a compound of the metal, most generally as an oxide of the metal. The metal may be used by itself or in various combinations of the metals or of their compounds. Other components may also be present as part of the photocatalyst. For example, dopants such as, for example, lanthanum, lithium, silver, and platinum, may be also be present. Electron transfer agents such as 1,4-dicyanobenzene, p-dicyanobenzene, chloanil, 1,4-dicyano-2,3,6-tetraethylbenzene, 1-cyanonaphthalene, 1,4,6-trinitrobenzene, hexamethylphosphoric triamide, methyl viologen dichloride hydrate, and the like, may be included as part of the photocatalyst system. See, for example, U.S. Pat. No. 5,720,858. Obviously, the amount of catalyst present in the photocatalytic reaction zone must be sufficient to effectively catalyze the reaction, that is to say it must be at least an effective catalytic amount.

The photocatalyst may be mixed with the reactants in the photocatalytic reaction zone, in which case it will be desirable to recover the catalyst from the product mixture for recycling back to the photocatalytic reaction zone. Since the catalyst is generally a particulate solid and the product mixture is a fluid, this may be accomplished by filtration or by use of a settling tank. However, in the present invention, it is preferred that the catalyst be immobilized in the photocatalytic reaction zone. As already noted, this is easily accomplished in the Holl-type mill reactor by simply coating one or both milling surfaces with the photocatalyst system. The relatively narrow photocatalytic reaction zone and high turbulence in the Holl-type mill reactor insure good contact between the photocatalyst and the reactants.

Unlike other synthetic routes for the production of methanol, the photocatalytic reaction between water and methane proceeds under relatively mild conditions. The reaction will proceed over a broad range of temperatures including room temperature up to the boiling point of water. However, temperatures above about 50° C. to about 99° C. are preferred, with temperatures in the range of from about 70° C. to about 90° C. being especially preferred. The reaction readily proceeds at atmospheric pressure, but generally there will be some super-atmospheric pressure in the reactor due to methane and water being continuously pumped into the reactor. In general, the pressure in the reactor will be in the range from about 1 atmosphere to about 20 atmospheres, with the range of from about 1 atmosphere to about 10 atmospheres being preferred. The reaction between methane and water takes place very rapidly. Therefore, residence time in the reaction zone need be very short, typically about 1 or 2 seconds is sufficient.

What is claimed is:

1. A photocatalytic method for the production of methanol from methane and water which comprises:
(a) forming an emulsion comprising a mixture of methane and water using a Holl-type mill which is characterized by a high shear treatment zone that is created by the opposing movement of two mill surfaces relative to one another wherein sub-Kolmogoroff eddies are formed in the space between said mill surfaces;
(b) exposing the emulsion of methane and water in a photocatalytic reaction zone to light in the presence of an effective catalytic amount of a photocatalyst under conditions suitable to support the conversion of methane and water to methanol, whereby the methane and water react to form methanol; and
(c) recovering methanol from the photocatalytic reaction zone.

2. The process of claim 1 wherein the photocatalytic reaction zone is located within the high shear treatment zone of the Holl-type mill.

3. The process of claim 1 wherein the conditions in the photocatalytic reaction zone include a temperature from about 50° C. to about 99° C. and a pressure in the range of from about 1 to about 20 atmospheres.

4. The process of claim 3 wherein the temperature is in the range of from about 70° C. to about 90° C. and the pressure is in the range of from about atmosphere to about 10 atmospheres.

5. The process of claim 1 wherein the photocatalyst contains a transition metal or a transition metal compound.

6. The process of claim 1 wherein the photocatalyst comprises at least one of molybdenum, vanadium, titanium, tungsten, or a compound of any of the aforesaid metals.

7. The process of claim 1 wherein the light is within the visible spectrum.

8. A photocatalytic method for the production of methanol from methane and water which comprises:
(a) forming an emulsion comprising a mixture of methane and water;
(b) exposing the emulsion of methane and water in a photocatalytic reaction zone to light in the presence of an effective catalytic amount of a photocatalyst under conditions suitable to support the conversion of methane and water to methanol, whereby the methane and water react to form methanol; and
(c) recovering methanol from the photocatalytic reaction zone.

9. The process of claim 8 wherein the emulsion of water and methane are maintained in a sufficiently thin layer that the light can penetrate its entire depth.

10. A reactor suitable for the photocatalytic production of methanol from methane and water which comprises:
(a) a Holl-type mill which is characterized by a high shear treatment zone that is created by the opposing movement of two mill surfaces relative to one another wherein sub-Kolmogoroff eddies are formed in the space between said mill surfaces, whereby an emulsion comprising methane and water will be formed, and wherein at least one of the said mill surfaces will allow the passage of light into the space between said mill surfaces;
(b) a light source positioned relative to at least one of said mill surfaces which admits light into the high shear treatment zone;
(c) an effective catalytic amount of a photocatalyst located in the high shear treatment zone;
(d) means for introducing methane and water into the high shear treatment zone; and
(e) means for withdrawing methanol from the high shear treatment zone.

11. The reactor of claim 10 wherein the two mill surfaces of the Holl-type mill comprise the inner surface of a hollow outer cylinder and the outer surface of an inner cylinder wherein the two cylinders are longitudinally positioned relative to one another such that the annular space formed between said inner surface of the hollow outer cylinder and said outer surface of the inner cylinder form the high shear treatment zone and the high shear is created by the rotation of at least one of the cylinders about its longitudinal axis relative to the other cylinder.

12. The reactor of claim 11 wherein the outer cylinder is stationary and the inner cylinder rotates within the outer cylinder.

13. The reactor of claim 11 wherein the photocatalyst is immobilized on at least one of the mill surfaces.

14. The reactor of claim 13 wherein the catalyst is immobilized on the outer surface of the inner cylinder.

15. The reactor of claim 13 wherein the catalyst is immobilized on the inner surface of the outer cylinder.

16. The reactor of claim 11 wherein the outer cylinder admits light into the high shear treatment zone.

17. The reactor of claim 11 wherein a light source is located within the inner cylinder and the inner cylinder admits light into the high shear treatment zone.

18. A reactor suitable for carrying out a photocatalytic reaction between reactants in order to form a product, wherein at least one liquid reactant and at least one gaseous reactant which is insoluble in said liquid are present, said reactor comprising:
(a) a Holl-type mill which is characterized by a high shear treatment zone that is created by the opposing movement of two mill surfaces relative to one another wherein sub-Kolmogoroff eddies are formed in the space between said mill surfaces, whereby an emulsion comprising the liquid reactant and the insoluble gaseous reactant will be formed, and wherein at least one of the said mill surfaces will allow the passage of light into the space between said mill surfaces;
(b) a light source positioned relative to at least one of said mill surfaces which admits light into the high shear treatment zone;
(c) an effective catalytic amount of a photocatalyst Immobilized within the high shear treatment zone;
(d) means for introducing a liquid reactant and a gaseous reactant into the high shear treatment zone; and
(e) means for withdrawing product from the high shear treatment zone.

19. The reactor of claim 18 wherein the two mill surfaces of the Holl-type mill comprise the inner surface of a hollow outer cylinder and the outer surface of an inner cylinder wherein the two cylinders are longitudinally positioned relative to one another such that the annular space formed between said inner surface of the hollow outer cylinder and said outer surface of the inner cylinder form the high shear treatment zone and the high shear is created by the rotation of at least one of the cylinders about its longitudinal axis relative to the other cylinder.

20. The reactor of claim 19 wherein the outer cylinder is stationary and the inner cylinder rotates within the outer cylinder.

21. The reactor of claim 20 wherein the catalyst is immobilized on the outer surface of the inner cylinder.

22. The reactor of claim 20 wherein the catalyst is immobilized on the inner surface of the outer cylinder.

23. The reactor of claim 19 wherein the light source is located outside of the outer cylinder and the outer cylinder admits light into the high shear treatment zone.

24. The reactor of claim 19 wherein a light source is located within the inner cylinder and the inner cylinder admits light into the high shear treatment zone.
